SEQUENCE LISTING (30 sequences)

SEQ ID NO: 1 - DNA, 46 bp, Artificial Sequence
  Synthetic oligonucleotide
cagccagccgcagcacgttcgctcataggagatatggtagagccgc

SEQ ID NO: 2 - DNA, 51 bp, Artificial Sequence
  Synthetic oligonucleotide
gagagaatttgtaccacctcccaccgggcacatagcagtcctagggacagt

SEQ ID NO: 3 - DNA, 22 bp, Artificial Sequence
  Synthetic oligonucleotide
ggcttggctctgctaacacgtt

SEQ ID NO: 4 - DNA, 22 bp, Artificial Sequence
  Synthetic oligonucleotide
ggacgtttgtaatgtccgctcc

SEQ ID NO: 5 - DNA, 20 bp, Artificial Sequence
  Synthetic oligonucleotide
acgtgctgcggctggctggt

SEQ ID NO: 6 - DNA, 48 bp, Artificial Sequence
  Synthetic oligonucleotide
ccaaagagtaaagtccttctctctcgagagactgttggcccttgaagg

SEQ ID NO: 7 - DNA, 47 bp, Artificial Sequence
  Synthetic oligonucleotide
gtgttgatgttatccaccttttgtggactaggaaaacagatcaatag

SEQ ID NO: 8 - DNA, 20 bp, Artificial Sequence
  Synthetic oligonucleotide
taatcctggaactccggtgc

SEQ ID NO: 9 - DNA, 23 bp, Artificial Sequence
  Synthesized oligonucleotide
tttatgccaattaacattttgac

SEQ ID NO: 10 - DNA, 24 bp, Artificial Sequence
  Synthetic oligonucleotide
cctccctgtggatgagagagaagg

SEQ ID NO: 11 - DNA, 52 bp, Artificial Sequence
  Synthetic oligonucleotide
cagccagccgcagcacgttcgctcataggagatatggtagagccgcrgacag

SEQ ID NO: 12 - DNA, 58 bp, Artificial Sequence
  Synthetic oligonucleotide
gagagaatttgtaccacctcccaccgggcacatagcagtcctagggacagtrggcgtt

SEQ ID NO: 13 - DNA, 29 bp, Artificial Sequence
  Synthetic oligonucleotide
ggcttggctctgctaacacgttrgctcaa

SEQ ID NO: 14 - DNA, 29 bp, Artificial Sequence
  Synthetic oligonucleotide
ggacgtttgtaatgtccgctccrggcact

SEQ ID NO: 15 - DNA, 20 bp, Artificial Sequence
  Synthetic oligonucleotide
acgtgctgcggctggctggt

SEQ ID NO: 16 - DNA, 54 bp, Artificial Sequence
  Synthetic oligonucleotide
ccaaagagtaaagtccttctctctcgagagactgttggcccttgaaggrgagca

SEQ ID NO: 17 - DNA, 53 bp, Artificial Sequence
  Synthetic oligonucleotide
gtgttgatgttatccaccttttgtggactaggaaaacagatcaatagrtaagc

SEQ ID NO: 18 - DNA, 26 bp, Artificial Sequence
  Synthetic oligonucleotide
taatcctggaactccggtgcraaggt

SEQ ID NO: 19 - DNA, 29 bp, Artificial Sequence
  Synthetic oligonucleotide
tttatgccaattaacattttgacrttatt

SEQ ID NO: 20 - DNA, 24 bp, Artificial Sequence
  Synthetic oligonucleotide
cctccctgtggatgagagagaagg

SEQ ID NO: 21 - DNA, 50 bp, Artificial Sequence
  Synthetic oligonucleotide
cagccagccgcagcacgttcgctcataggagatatggtagagccgcrgag

SEQ ID NO: 22 - DNA, 55 bp, Artificial Sequence
  Synthetic oligonucleotide
gagagaatttgtaccacctcccaccgggcacatagcagtcctagggacagtrgtt

SEQ ID NO: 23 - DNA, 26 bp, Artificial Sequence
  Synthetic oligonucleotide
ggcttggctctgctaacacgttrcaa

SEQ ID NO: 24 - DNA, 26 bp, Artificial Sequence
  Synthetic oligonucleotide
ggacgtttgtaatgtccgctccrgct

SEQ ID NO: 25 - DNA, 20 bp, Artificial Sequence
  Synthetic oligonucleotide
acgtgctgcggctggctggt

SEQ ID NO: 26 - DNA, 52 bp, Artificial Sequence
  Synthetic oligonucleotide
ccaaagagtaaagtccttctctctcgagagactgttggcccttgaaggrgca

SEQ ID NO: 27 - DNA, 51 bp, Artificial Sequence
  Synthetic oligonucleotide
gtgttgatgttatccaccttttgtggactaggaaaacagatcaatagrtgc

SEQ ID NO: 28 - DNA, 24 bp, Artificial Sequence
  Synthetic oligonucleotide
taatcctggaactccggtgcragt

SEQ ID NO: 29 - DNA, 27 bp, Artificial Sequence
  Synthetic oligonucleotide
tttatgccaattaacattttgacrttt

SEQ ID NO: 30 - DNA, 24 bp, Artificial Sequence
  Synthetic oligonucleotide
cctccctgtggatgagagagaagg

(19) United States
(12) Patent Application Publication
(10) Pub. No.: US 2017/0218438 A1
(43) Pub. Date: Aug. 3, 2017

(54) CLEAVABLE PRIMERS FOR ISOTHERMAL AMPLIFICATION

(71) Applicant: Integrated DNA Technologies, Inc., Coralville, IA (US)

(72) Inventors: Joseph Dobosy, Coralville, IA (US); Aurita Menezes, Morrisville, NC (US); Caifu Chen, Palo Alto, CA (US); Mark Behlke, Coralville, IA (US)

(73) Assignee: Integrated DNA Technologies, Inc., Coralville, IA (US)

(21) Appl. No.: 15/421,577

(22) Filed: Feb. 1, 2017

Related U.S. Application Data
(60) Provisional application No. 62/289,400, filed on Feb. 1, 2016.

(51) Int. Cl. C12Q 1/68; C12N 9/22; C12N 9/12

(52) U.S. Cl. CPC C12Q 1/6853; C12N 9/1252; C12N 9/22; C12Y 207/07007; C12Y 301/26004; C12Q 1/6848

(57) ABSTRACT

The invention provides a more efficient and less error-prone method of performing LAMP. The invention also provides a method for utilizing an RNase H2-cleavable probe as a technique for generating signal from the reaction, potentially increasing the specificity of the signal generation.

### FIELD OF THE INVENTION

This invention pertains to the use of cleavable primers in isothermal amplification.

### BACKGROUND OF THE INVENTION

The amplification of nucleic acids is a widely used tool in the life sciences. One of the most widely used methods is polymerase chain reaction (PCR). Conventional PCR requires a heating step to denature hybridized DNA, and therefore requires a heating mechanism such as a heat block that would limit its applicability to smaller clinical labs or field applications.

Amplification methods have been developed that do not require a change in reaction temperature, and they are generally referred to as isothermal amplification methods. They not only don't require complex heating/cooling elements, but they also are typically faster than traditional PCR. Examples include helicase-dependent amplification (HDA), nicking-enzyme amplification reaction (NEAR), strand displacement amplification (SDA), and loop-meditated isothermal amplification (LAMP).

LAMP requires a minimum of four different primers designed to recognize six different regions of the desired amplicon (Notomi, et al. Nucleic Acids Research, 28(12) (2000)). The amplification reaction depends on the strand displacement activity of the DNA polymerase, usually from Bacillus stearothermophilus (Bst). The products have a structure that consists of a long chain of inverted repeats of the target sequence.

LAMP methods are prone to the formation of primer-dimers due to large number of primers and the mesophilic DNA polymerase. This combination can cause primer-dimers to form rapidly when incubated even for a short time at room temperature (<2 hours) (see Tanner and Evans, U.S. Patent App No. US20130122551). U.S. Application 2013/0122551 teaches designs for unmodified LAMP primers that can form primer-dimers. The '551 application describes the use of a single-stranded binding (SSB) protein to address the same issue of primer-dimers.

LAMP also lacks a 5′→3′ exonuclease activity in the amplifying BST polymerase, due to the fact that this activity would destroy the amplification by competing with the essential strand-displacement activity.

The invention provides such methods and composition to reduce primer-dimers and offer increased specificity. These and other advantages of the invention, as well as additional inventive features, will be apparent from the description of the invention provided herein.

### BRIEF SUMMARY OF THE INVENTION

The invention provides a more efficient and less error-prone method of performing LAMP that utilizes RNase H-cleavable primers. The invention also provides a method for utilizing an RNase H-cleavable probe as a technique for generating signal from the reaction, potentially increasing the specificity of the signal generation.

In one embodiment, an assay preparation is provided, the preparation comprising a thermostable polymerase, at least four distinct rhPrimers, an RNase H enzyme and a buffer. In a further embodiment, the RNase H enzyme is RNase H2.

In another embodiment, an assay preparation is provided, the preparation comprising a thermostable polymerase, at least four distinct oligonucleotide primers, an RNase H-cleavable probe (“rhProbe”), RNase H enzyme and a buffer. In a further embodiment, the at least four distinct oligonucleotide primers are rhPrimers. In a further embodiment, the RNase H enzyme is RNase H2.

In another embodiment, the rhProbe and/or the rhPrimers used to reduce or eliminate the formation of primer-dimers in a LAMP reaction comprise a cleavage domain with a cleavage site, located within or adjacent to the cleavage domain, which is cleavable by an RNase H2 enzyme, wherein said cleavage site is positioned 5′ of a blocking group at or near the 3′-end of the rhPrimer or rhProbe which prevents primer extension and/or PCR, and wherein said cleavage domain includes a sequence 5′ to 3′ Rx or RDx where R is an RNA residue, D is a DNA residue and x is an abasic residue blocking group or a detectable label.

In a further embodiment, the 3′ cleavable regions of the rhPrimers or rhProbes are comprised of rDDDDx, wherein the “r” is at least one RNA base, each “D” represents a DNA base, and the “x” is an abasic residue. In another embodiment, the 3′ cleavable regions of the rhPrimers are comprised of rDDDDMx, wherein the “r” is at least one RNA base, each “D” represents a DNA base, the “M” represents a mismatch to the target, and the “x” is an abasic residue or a detectable label. For rhProbes, the cleaved region contains a detectable label, either at “x” or elsewhere within the cleaved region.

In another embodiment, the 3′ cleavable regions of the rhPrimers or rhProbes are comprised of rDxxD or rDxxDM. For rhProbes, the cleavable region would contain a detectable label at the 3′ terminus or at another position within the cleaved region.

### DETAILED DESCRIPTION OF THE INVENTION

The proposed method involves novel methods and compositions to improve LAMP. In one embodiment the methods utilize blocked primers that are utilized in the LAMP methods. In a further embodiment, the methods utilize blocked primers that are activated when cleaved with RNase H (“rhPrimers”) (see Behlke et al., U.S. Pat. No. 8,911,948, incorporated herein in its entirety), thereby made functional for use in LAMP assays. When the rhPrimers are blocked and inactive, they are unable to function in primer-dimer side reactions.

In another embodiment, the rhPrimers used to reduce or eliminate the formation of primer-dimers in a LAMP reaction comprise a cleavage domain with a cleavage site, located within or adjacent to the cleavage domain, which is cleavable by an RNase H2 enzyme, wherein said cleavage site is positioned 5′ of a blocking group at or near the 3′-end of the oligonucleotide primer which prevents primer extension and/or PCR, and wherein said cleavage domain includes a sequence 5′ to 3′ Rx or RDx where R is an RNA residue, D is a DNA residue and x is an abasic residue blocking group. In a further embodiment, the cleavable region design is rDDDDMx (Gen1) or rDxxDM (Gen 2) primer designs. The r is an RNA residue, D is a DNA residue, x is an abasic residue and M is a mismatch. The mismatch is an optional feature.

In another embodiment, a LAMP assay utilizes an RNase H2 cleavable probe (“rhProbe”) that can reduce or eliminate the detection of primer-dimer signal in LAMP reactions. This may be done in conjunction with blocked-cleavable rhPrimers, or it may be done with conventional unmodified primers.

To overcome the lack of the 5′->3′exonuclease activity of the polymerase, a probe containing a single or multiple RNA bases that can be cleaved with RNase H2 could be used.

The following examples further illustrate the invention but, of course, should not be construed as in any way limiting its scope.

### EXAMPLE 1

This example outlines a method of demonstrating the use of rhPrimers in a LAMP reaction to reduce primer-dimer formation.

To evaluate the functionality of rhPrimers for LAMP protocols, three assays could be designed using (1) unmodified control primers; “Gen1” rDDDDMx primers wherein “r” is an RNA base, “D” is a DNA base, “m” is a mismatch and “x” is a C3 spacer; and “Gen2” rDxxDM primers. The details of the assays to be used in the evaluations are detailed in Tables 1, 2 and 3. LAMP reactions utilizing each type of primer will be held at 25° C. (room temperature) for 0 or 2 hours prior to testing. This will allow for the formation of the primer-dimer products. After the room temperature hold, all reactions will be run at 65° C. for 2 hours in a BioRad CFX384 or Roche LightCycler 480. Signal generation in all of these reactions will be performed with 1× EvaGreen, added to the reaction (see: Biotechnology Letters, December 2007, Volume 29, Issue 12, pp 1939-1946).

TABLE 1
Controlled unmodified primer designs

| SEQ | | |
|---|---|---|
| ID | | |
| No. | Name | Sequence |
| 1 | FTP- | CAGCCAGCCGCAGCACGTTCGCTCATAGGAGATATGGTA |
| | lambda | GAGCCGC |
| 2 | BIP- | GAGAGAATTTGTACCACCTCCCACCGGGCACATAGCAGT |
| | lambda | CCTAGGGACAGT |
| 3 | E3- | GGCTTGGCTCTGCTAACACGTT |
| | lambda | |
| 4 | B3- | GGACGTTTGTAATGTCCGCTCC |
| | lambda | |
| 5 | rhLAMP | FAM-ACGTGCTGCGgCTGGCTGGT-IBFQ |
| | Probe- | |
| | lambda | |
| 6 | FTP- | CCAAAGAGTAAAGTCCTTCTCTCTCGAGAGACTGTTGGC |
| | hCFTR | CCTTGAAGG |
| 7 | BIP- | GTGTTGATGTTATCCACCTTTTGTGGACTAGGAAAACAG |
| | hCFTR | ATCAATAG |
| 8 | E3- | TAATCCTGGAACTCCGGTGC |
| | hCFTR | |
| 9 | B3- | TTTATGCCAATTAACATTTTGAC |
| | hCFTR | |
| 10 | rhLAMP | FAM-CCTCCCTGTGGATgAGAGAGAAGG-IBFQ |
| | Probe- | |
| | hCFTR | |

DNA bases are uppercase; RNA bases are lowercase. FAM=6-carboxyfluorescein; IBFQ=Iowa Black™ FQ fluorescence quencher

TABLE 2
Gen1 primer designs

| SEQ | | |
|---|---|---|
| ID | | |
| No. | Name | Sequence |
| 11 | FTP- | CAGCCAGCCGCAGCACGTTCGCTCATAGGAGATATGGTAG |
| | lambda | AGCCGCrAGACAG/3SpC3/ |
| 12 | BIP- | GAGAGAATTTGTACCACCTCCCACCGGGCACATAGCAGTC |
| | lambda | CTAGGGACAGTrGGCGTT/3SpC3/ |
| 13 | E3- | GGCTTGGCTCTGCTAACACGTTrGCTCAA/3SpC3/ |
| | lambda | |
| 14 | B3- | GGACGTTTGTAATGTCCGCTCCrGGCACT/3SpC3/ |
| | lambda | |
| 15 | rhLAMP | /56-FAM/ACGTGCTGCGgCTGGCTGGT/3IABkFQ/ |
| | Probe- | |
| | lambda | |
| 16 | FTP- | CCAAAGAGTAAAGTCCTTCTCTCTCGAGAGACTGTTGGCC |
| | hCFTR | CTTGAAGGrAGAGCA/3SpC3/ |
| 17 | BIP- | GTGTTGATGTTATCCACCTTTTGTGGACTAGGAAAACAGA |
| | hCFTR | TCAATAGrATAAGC/3SpC3/ |
| 18 | E3- | TAATCCTGGAACTCCGGTGCrUAAGGT/3SpC3/ |
| | hCFTR | |
| 19 | B3- | TTTATGCCAATTAACATTTTGACrUTTATT/3SpC3/ |
| | hCFTR | |
| 20 | rhLAMP | /56-FAM/CCTCCCTGTGGATgAGAGAGAAGG/3IABkFQ/ |
| | Probe- | |
| | hCFTR | |

DNA bases are uppercase; RNA bases are lowercase. FAM=6-carboxyfluorescein; IBFQ=Iowa Black™ FQ fluorescence quencher. 3 SpC3=C3 spacer

TABLE 3
GEN2 primer designs

| SEQ | | |
|---|---|---|
| ID | | |
| No. | Name | Sequence |
| 21 | FTP- | CAGCCAGCCGCAGCACGTTCGCTCATAGGAGATATGGTAG |
| | lambda | AGCCGCrAG/iSpC3//iSpC3/AG |
| 22 | BIP- | GAGAGAATTTGTACCACCTCCCACCGGGCACATAGCAGTC |
| | lambda | CTAGGGACAGTrGG/iSpC3//iSpC3/TT |
| 23 | E3- | GGCTTGGCTCTGCTAACACGTTrGC/iSpc3//iSpC3/ |
| | lambda | AA |
| 24 | B3- | GGACGTTTGTAATGTCCGCTCCrGG/iSpC3//iSpC3/ |
| | lambda | CT |
| 25 | rhLAMP | /56-FAM/ACGTGCTGCGgCTGGCTGGT/3IABkFQ/ |
| | Probe- | |
| | lambda | |
| 26 | FTP- | CCAAAGAGTAAAGTCCTTCTCTCTCGAGAGACTGTTGGCC |
| | hCFTR | CTTGAAGGrAG/iSpC3//iSpC3/CA |
| 27 | BIP- | GTGTTGATGTTATCCACCTTTTGTGGACTAGGAAAACAGA |
| | hCFTR | TCAATAGrAT/iSpC3//iSpC3/GC |
| 28 | E3- | TAATCCTGGAACTCCGGTGCrUA/iSpC3//iSpC3/GT |
| | hCFTR | |
| 29 | B3- | TTTATGCCAATTAACATTTTGACrUT/iSpC3//iSpC3/ |
| | hCFTR | TT |
| 30 | rhLAMP | /56-FAM/CCTCCCTGTGGATgAGAGAGAAGG/ |
| | Probe- | 3IABkFQ/ |
| | hCFTR | |

DNA bases are uppercase; RNA bases are lowercase. FAM=6-carboxyfluorescein; IBFQ=Iowa Black™ FQ fluorescence quencher. 3SpC3=C3 spacer

Each assay could be performed as follows:

The samples will either be Coriell gDNA or Lambda phage genomic DNA. Each assay condition could be run in triplicate with sample input of 5 ng Lambda phage genomic DNA or 20 ng human genomic DNA. For each assay, reactions could run using unmodified primers with intercalating dye (e.g., EvaGreen) and cleavable, blocked primers with an intercalating dye. For each assay, reactions will be run using zero, or titrated levels of RNase H2.

For unmodified, unblocked primers, the titration will include 0 mU, 0.6 mU, 2.6 mU, 5 mU, 25 mU, and 100 mU/reaction. For the second set of primers, the titration will include 0 mU, 0.6 mU, 2.6 mU, 5 mU, 25 mU, and 100 mU/reaction. For the third set of primers, the titration will include 0 mU, 2.6 mU, 25 mU, 100 mU, 200 mU, and 400 mU/reaction.

Unmodified assays containing 2 ug Extreme Thermostable (ET) SSB (NEB: https://www.neb.com/products/m2401-et-ssb) will also be performed as a positive control, and for comparison with blocked cleavable primers.

Each assay will be run in triplicate. Comparisons between the unmodified LAMP assays and the modified LAMP assays would be performed, by comparing the length of time required for formation of signal-generating products. It is expected that the unblocked primers in the LAMP reactions will produce these products significantly later than the reactions containing the blocked rhPrimers. The control reactions containing the unblocked primers and the SSB protein will also be compared with the rhPrimer reactions, and are expected to give nearly identical amplification rates, indicating that both systems can reduce the formation of dimers.

The 25 μL EvaGreen reaction mixtures will include:
12.5 μL of 2× Master Mix (1× is 20 mM Tris pH 8.8 @25° C., 10 mM KCl, 10 mM
(NH4)2SO4, 8 mM MgSO4, 0.01% Tween-20, 1.4 mM dNTPs.)
1.6 μM FIP primer
1.6 μM BIP primer
1× EvaGreen dye
0.2 μM F3 primer
0.2 μM B3 primer
8 U BST DNA Polymerase (New England Biolabs: https://www.neb.com/products/m0275-bst-dna-polymerase-large-fragment)
1 μL of RNase H2 (for no RNase H2 control, buffer D will be used)
Nuclease Free Water to 25 uL
2 μL sample (either 10 ng/μL human gDNA or 2.5 ng/uL lambda genomic DNA)+/−2 ug ET SSB

### EXAMPLE 2

The following example outlines a method of demonstrating the use of RNase H2 cleavable probes in a LAMP reaction.

To evaluate the functionality of an RNase H2 cleavable probe for LAMP protocols, and whether it can be used in conjunction with blocked primers, three assays will be initially evaluated—designed using unmodified control primers, GEN1 (rDDDDmx) primers, or GEN2 (rDxxDm) primers, each design to be used with an RNase H2 cleavable probe. The details of the assays to be used in the evaluations are detailed in Tables 1, 2 and 3. LAMP reactions utilizing each type of primer will be held at 25° C. (room temperature) for 0 or 2 hours prior to testing. This will allow for the formation of the primer-dimer products. After the room temperature hold, all LAMP reactions will be run at 65° C. for 2 hours in a BioRad CFX384 or Roche LightCycler 480.

Each assay will be performed as follows: the samples will either be Coriell gDNA or Lambda phage genomic DNA. Each assay condition will be run in triplicate with sample input of 5 ng Lambda phage genomic DNA or 20 ng human genomic DNA. For each assay, reactions will run using unmodified primers with RNase H2 cleavable probe and cleavable, blocked primers with RNase H2 cleavable probe

For each assay, reactions will be run using zero, or titrated levels of RNase H2. For unmodified, unblocked primers, the titration will include 0 mU, 0.6 mU, 2.6 mU, 5 mU, 25 mU, and 100 mU/reaction. For Gen1 primers, the titration will include 0 mU, 0.6 mU, 2.6 mU, 5 mU, 25 mU, and 100 mU/reaction. For Gen2 primers, the titration will include 0 mU, 2.6 mU, 25 mU, 100 mU, 200 mU, and 400 mU/reaction.

Unmodified assays containing 2 ug Extreme Thermostable (ET) SSB (NEB: https://www.neb.com/products/m2401-et-ssb) will also be performed as a positive control, and for comparison with blocked cleavable primers.

Each assay will be run in triplicate. The 25 μL probe reaction mixtures will include:
12.5 μL of 2× Master Mix (1× is 20 mM Tris pH 8.8 @25° C., 10 mM KCl, 10 mM (NH4)2SO4, 8 mM MgSO4, 0.01% Tween-20, 1.4 mM dNTPs.)
1.6 μM FIP primer
1.6 μM BIP primer
0.2 μM RNase H2 cleavable probe
0.2 μM F3 primer
0.2 μM B3 primer
8 U BST DNA Polymerase (New England Biolabs: https://www.neb.com/products/m0275-bst-dna-polymerase-large-fragment)
1 μL of RNase H2 (for no RNase H2 control, buffer D will be used) Nuclease Free Water to 25 uL
2 μL sample (either 10 ng/μL human gDNA or 2.5 ng/uL lambda genomic DNA)+/−2 ug ET SSB

It is expected that the signal detection will only occur in the reactions that contain RNase H2. It is expected that the unblocked primers in the LAMP reactions will produce these products significantly later than the reactions containing the blocked rhPrimers. The control reactions containing the unblocked primers and the SSB protein will also be compared with the rhPrimer reactions, and are expected to give nearly identical amplification rates, indicating that both systems can reduce the formation of dimers.

Adding a 3′ hairpin domain could further reduce the formation of dimers in LAMP reactions. In this embodiment, the hairpin would form a duplex on the 3′-end that changes the conformation of the cleavable domain in a manner that limits or eliminates the cleaving enzyme to actually cleave the domain. For example, the hairpin could form a double-stranded complex that is adjacent to the RNA base, or it could extend further to hybridize to the RNA base but still not form a double-stranded region that would be recognized by RNase H.

In all of these reactions, the cleavable domain is an RNA, but any cleavable nucleotide is considered. RNA residues to include a fluorine at the 2′ position in particular could be utilized for this procedure, but other modifications, including 2′OMe, 2′ chloro, locked nucleic acids, and other modifications are also potential cleavage domains.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A preparation, comprising: a thermostable polymerase; at least four different rhPrimers, and RNase H2 enzyme and a buffer.

2. The rhPrimers of claim 1 wherein the rhPrimers comprise a cleavage domain with a cleavage site, located within or adjacent to the cleavage domain, which is cleavable by an RNase H2 enzyme, wherein said cleavage site is positioned 5′ of a blocking group at or near the 3′-end of the oligonucleotide primer which prevents primer extension and/or PCR, and wherein said cleavage domain includes a sequence 5′ to 3′ Rx or RDx where R is an RNA residue, D is a DNA residue and x is an abasic residue blocking group.

3. The rhPrimers of claim 2 wherein the sequence is RDDDDx, RDDDDMx, RDxxD, or RDxxDM, wherein M is a mismatch.

4. The rhPrimers of claim 2 where the sequence contains a 3′ hairpin domain either to prevent premature primer deblocking, improve mismatch discrimination, or enhance primer-dimer prevention.

5. A preparation, comprising: a thermostable polymerase; at least four different oligonucleotide primers, and RNase H2 enzyme, and RNase H2 cleavable probe and a buffer.

6. The preparation from claim 4 wherein the at least four distinct oligonucleotide primers are rhPrimers.

7. The rhPrimers of claim 4 wherein the rhPrimers comprise a cleavage domain with a cleavage site, located within or adjacent to the cleavage domain, which is cleavable by an RNase H2 enzyme, wherein said cleavage site is positioned 5′ of a blocking group at or near the 3′-end of the oligonucleotide primer which prevents primer extension and/or PCR, and wherein said cleavage domain includes a sequence 5′ to 3′ Rx or RDx where R is an RNA residue, D is a DNA residue and x is an abasic residue blocking group.
